(19) United States
(12) Plant Patent Application Publication
(10) Pub. No.: US 2009/0293164 P1
(43) Pub. Date: Nov. 26, 2009

(54) St. Augustinegrass plant named 'Eclipse'

(50) ;

(76) Inventors: H. Wayne Philley, Mathiston, MS (US); Jeffrey V. Krans, Manitowish, WI (US); Clarence E. Watson, Stillwater, OK (US)

Correspondence Address: Larry A. Schemmel;Office of the Attorney General, P.O. Box 1850, Jackson, MS, 39215-1850, US

(21) Appl. No.: 12/154,431

(22) Filed: May 22, 2008

(51) Int. Cl. A01H 5/00

(52) U.S. Cl. PLT/392

(57) ABSTRACT

St. Augustinegrass plant Eclipse is a new and distinct variety of perennial St. Augustinegrass cultivar, characterized by its short and narrow leaf blades, fine leaf texture, short internode length and diameter, and superior turf quality and particularly turf density when grown under shade or dense shade. Eclipse is also distinguished by its genetic color and fall and winter color characteristics.

### LATIN NAME

Stenotaphrum secundatum

### VARIETAL DENOMINATION

Eclipse

### BACKGROUND OF THE INVENTION

This invention relates to a new and distinct perennial variety of St. Augustinegrass that is well-suited for turfgrass applications. It is a high-quality, high-density cultivar well-adapted for warm weather climates similar to that found in southern climates where high quality St. Augustinegrass grass cultivar varieties have previously not been available. The Latin name of the genus and species of the new cultivar disclosed herein is Stenotaphrum secundatum. This novel hybrid genotype has been given the varietal denomination ‘Eclipse’ and is a perennial, asexually propagated genotype of St. Augustinegrass, which typically grows vigorously well in warm weather climates and spreads through creeping stolons that root at the nodes contacting soil with adequate moisture. Commonly-known varieties of this genus and species include Raleigh St. Augustine (not patented), Floratam (not patented), B12 (U.S. Plant Pat. No. 16,174 and marketed under the tradename Sapphire™), and SS-100 (U.S. Plant Pat. No. 9,395 and marketed under the tradename Palmetto™). The designation Eclipse was evaluated under the experimental name MSA 31 and may also designate this particular plant in commerce. It is expected that the plant of this invention will be marketed under the synonym Eclipse as a tradename. This plant is identified interchangeably by its designation and its experimental name throughout this disclosure and its tables and figures. This high quality novel and distinct variety of St. Augustinegrass was vegetatively propagated at Starkville, Miss.

### SUMMARY OF THE INVENTION

The cultivar Eclipse (experimental name MSA 31) is a new and distinctive variety of St. Augustinegrass characterized by its unique pedigree and very good shade tolerance and high turf quality. The traits of the invention are continually maintained when propagated asexually. This new variety provides an excellent appealing uniform, dense, dark green turf at locations where other St. Augustinegrasses are weakened by excessive shade and disease injury. Eclipse exhibits other excellent qualities and characteristics such as turf density, fine leaf texture, fall and winter color, fast spring green up at warm sites, and excellent low seedhead ratings compared to other St. Augustinegrass cultivars which allow it to be further distinguished from other cultivars.

### BRIEF DESCRIPTION OF THE DRAWINGS

FIG. 1 is a flow chart diagram and graphical illustration of the unique pedigree of Eclipse showing the crossing of St. Augustinegrass genotypes resulting in the distinctive new cultivar.

FIG. 2 is a color photograph taken on Apr. 24, 2006 at the Mississippi State University Department of Plant & Soil Sciences Dorman greenhouses of stolon segments of four (4) St. Augustinegrass cultivars that compares and illustrates the distinct morphologies of each segment. From left to right, Floratam with its long leaves and purple internodes is shown on the far left. Next in line to the right is Raleigh with its long leaves and green/yellow internodes. Next in line to the right is Aurora (experimental name MSA 2-3-98) with its small leaves and green internodes. Finally on the far right, Eclipse is shown with its small leaves and dark green internodes.

FIG. 3 is a color photograph taken on Apr. 24, 2006 at the Mississippi State University Department of Plant & Soil Sciences Dorman greenhouses of four (4) whole non-mown St. Augustinegrass cultivar plants in growing pots showing, from left to right: Floratam with its longer leaves, tall stature, and purple/red internodes; Raleigh with its longer leaves, tall stature, and green/yellow internodes; Aurora with its smaller leaves, shorter stature, and more green internodes; and Eclipse with its small leaves, shorter stature, and darker green internodes.

FIG. 4 is a color photograph taken on Oct. 26, 2004 at the Mississippi State University Plant Science Research Center in Starkville, Miss. during the 2002 NTEP test showing the turf density of the Eclipse St. Augustinegrass cultivar.

FIG. 5 is a color photograph taken on Oct. 26, 2004 at the Mississippi State University Plant Science Research Center in Starkville, Miss. during the 2002 NTEP test showing field plots of six (6) cultivars (1 full replication), specifically depicting Raleigh in the left foreground, Eclipse St. Augustinegrass in the left center, Mercedes in the left background, Delmar in the right foreground, Floratam in the right center, and Aurora in the right background.

FIG. 6 is a photograph of the unique DNA amplification profile of Eclipse and the DNA profiles of five (5) other St. Augustinegrass cultivars using RAPD primer OPAC20, illustrating that Eclipse is unique and different from Sapphire™, Raleigh, Aurora, Palmetto™, and Floratam.

FIG. 7 is a photograph of the unique DNA amplification profile of Eclipse and the DNA profiles of five (5) other St. Augustinegrass cultivars using RAPD primer OPAU1, illustrating that Eclipse is unique and different from Sapphire™, Raleigh, Aurora, Palmetto™, and Floratam.

FIG. 8 is a photograph of the unique DNA amplification profile of Eclipse and the DNA profiles of five (5) other St. Augustinegrass cultivars using RAPD primer OPBA9, illustrating that Eclipse is unique and different from Sapphire™, Raleigh, Aurora, Palmetto™, and Floratam.

FIG. 9 is a photograph of the unique DNA amplification profile of Eclipse and the DNA profiles of five (5) other St. Augustinegrass cultivars using RAPD primer OPM5, illustrating that Eclipse is unique and different from Sapphire™, Raleigh, Aurora, Palmetto™, and Floratam.

### DETAILED BOTANICAL DESCRIPTION OF THE VARIETY

The following is a detailed botanical description of the characteristics of the new Stenotaphrum secundatum grass variety known as Eclipse (experimental name MSA 31), based upon observations of the plant grown under conventional greenhouse conditions and in nursery pots and field plots at the Mississippi State University (MSU) Mississippi Agricultural and Forestry Experiment Station in Oktibbeha County, Miss. Color notations of plant tissues are based upon the Munsell® Color Chart for plant tissues [Munsell Book of Color: Glossy Finish Edition, Munsell Color, Baltimore, Md., 1976; and Munsell Color Charts for Plant Tissues, Munsell Color, Baltimore, Md., 1977]. Color notations are affected by light quality and fertility and general plant growth. Certain characteristics will vary depending on the age of the plants, such that characteristics such as dimensions, sizes, and colors are approximations or averages since the variety has not been observed under every possible environmental condition. Therefore, the phenotype of the variety may differ from the descriptions depending upon environmental variations including, but not limited to, the season, temperatures, day lengths, light direction and quality, and fertilization, as well as other factors.

Eclipse is a high quality, high density, perennial, vegetatively (asexually) propagated genotype of St. Augustinegrass. It is well-adapted for turf applications in southern and warmer locations typically where Floratam, the most popular St. Augustinegrass cultivar, is used. Eclipse is capable of sustaining high turf quality and excellent density ratings in shade. The creeping or ascendant stolons of Eclipse are dorsiventrally compressed and root adventitiously at the nodes. Its leaf blade color is dark green and is rated 7.5 GY 4/4 for field-grown plots and 7.5 GY 4/4 to 4/6 for greenhouse-grown pots, both ratings based on the Munsell® Color Chart for plant tissue. The color chart match was completed using natural sunlight with no supplemental lighting for the Eclipse cultivars in the field-grown plots and the greenhouse-grown pots, both in the month of May. Eclipse has a green internode color that is fairly stable, in contrast to the internodes of other St. Augustinegrass cultivars that may change to various shades of purple with cooler temperatures, such as Floratam in particular. Eclipse typically has yellow anthers and white stigmas and has an unreduced chromosome number of 18.

The leaf texture of Eclipse is finer than Raleigh and Floratam St. Augustinegrasses. Eclipse has better cold resistance or tolerance than Floratam but less than Raleigh, typically considered the cold tolerance standard. Eclipse is recommended for U.S. Department of Agriculture (USDA) plant hardiness zones 8 and higher, but not necessarily recommended for sites or locations where winterkill of St. Augustinegrasses is a problem.

### Unique Pedigree and Origin of Eclipse

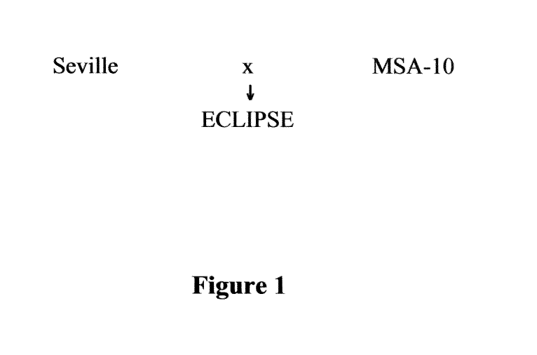

Eclipse St. Augustinegrass cultivar resulted from a cross of two diploid St. Augustinegrass plants selected for their specific superior qualities. Eclipse resulted from an F1 plant (F1 refers to first generation) cross between Seville St. Augustinegrass (U.S. Plant Pat. No. 4,097, issued Sep. 6, 1977, now expired) and MSA-10, an experimental cultivar selected for cold tolerance at Mississippi State University. Six (6) plants were produced from this cross. Eclipse was selected over all its siblings for its high turf density and quality. The original, single seed that produced Eclipse was germinated in a temperature-controlled germination chamber. The seedling was transplanted to a pot grown in a greenhouse and eventually transplanted to a field nursery. From that point in time forward, Eclipse has been vegetatively propagated and has consistently and completely been stably reproduced by the aforementioned asexual propagation. FIG. 1 shows the pedigree of Eclipse in a flowchart format. In FIG. 1, Seville is a highly fertile, semi-dwarf diploid cultivar selected for its high density and fine leaf texture but not necessarily for its cold tolerance. ‘MSA-10’ is a Mississippi St. Augustinegrass experimental cultivar from Mississippi State University (MSU) selected for cold tolerance and winter survival. The unique origin of Eclipse shown distinguishes it from other commercially produced St. Augustinegrass varieties in that most of such varieties are of unknown origin. The pedigree of Eclipse is unique in that its parents are known. As a result, Eclipse has higher turf quality and higher turf density, particularly in shade and in dense tree shade, than either of its ancestors.

### Morphology of Eclipse

The morphological characteristics of Eclipse also distinguish it from other St. Augustinegrass cultivars. To measure morphological characteristics, five (5) four-inch plugs of each of four (4) St. Augustinegrass cultivars were harvested from established field plots at Starkville, Miss. in March, 2006. These twenty (20) plugs were washed free of soil and transplanted to six-inch pots filled with commercial potting mix. These twenty (20) plugs were grown non-mown in a greenhouse for six (6) weeks before measurements were initiated. All stolons that grew out from the original plug were systematically evaluated, beginning with the youngest visible internode and proceeding backward toward the pot. Measurements included diameter and length of all internodes and width and length of all leaf blades.

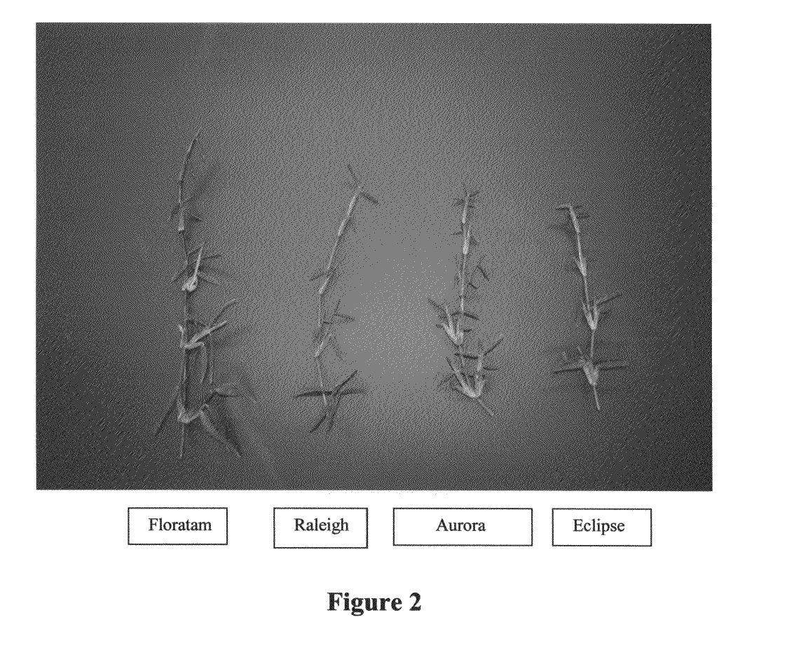

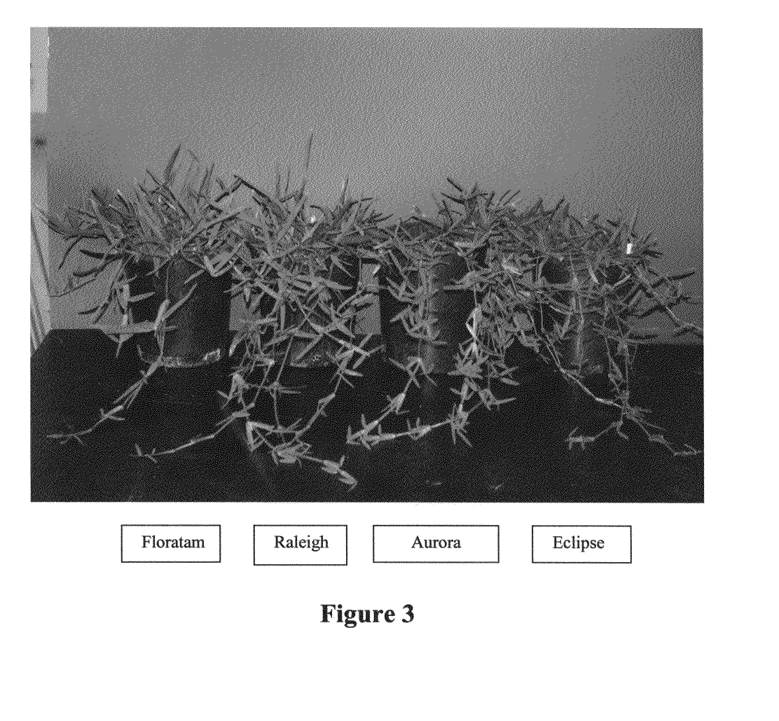

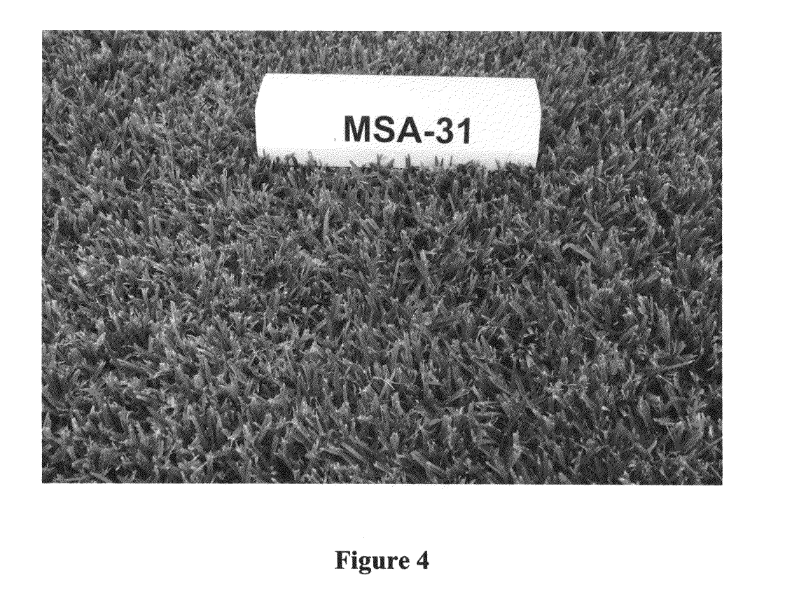

The non-mown leaf blade length of Eclipse was significantly shorter than that of cultivars Floratam and Raleigh but similar to MSU experimental cultivar MSA 2-3-98 (Aurora) (Table 1 and FIG. 2). FIG. 2 shows stolons of four (4) St. Augustinegrass cultivars that illustrate different or comparative morphologies, from left to right: Floratam, Raleigh, Aurora, and Eclipse. FIG. 3 shows the same four cultivars as whole non-mown plants in growing pots and shows the smaller morphology of Eclipse. FIG. 4 shows a close-up photograph of the 2002 NTEP test plot of Eclipse and, specifically, Eclipse's excellent density characteristics. The leaf blade width of Eclipse was significantly narrower than Floratam and Raleigh and not significantly wider than Aurora. The internode length of Eclipse was significantly shorter than Floratam, Raleigh, and Aurora. The internode diameter of Eclipse was significantly smaller than Floratam, Raleigh, and Aurora. (Table 1). Eclipse also differs visually from other cultivars in other respects. For example, Eclipse's yellow anthers and white stigmas are similar to certain cultivars, such as the white stigmas of Raleigh and Palmetto™, but different from Palmetto™'s and Floratam's orange-yellow anthers, Floratam's purple stigmas, and Sapphire™'s gray-orange anthers and purple-violet stigmas.

TABLE 1
Morphology measurements of St. Augustinegrass
cultivars grown in non-mown pots.

| | Leaf Blade | Leaf Blade | Internode | Internode |
|---|---|---|---|---|
| Cultivar | Length mm | Width mm | Length mm | Diameter mm |
| Floratam | 63.9 | 8.9 | 62.2 | 3.54 |
| Raleigh | 34.3 | 7.6 | 67.1 | 3.11 |
| Aurora | 27.9 | 6.6 | 53.0 | 2.98 |
| Eclipse | 27.9 | 6.5 | 49.0 | 2.48 |
| LSD (0.05) | 4.1 | 0.3 | 2.9 | 0.06 |

### Turf Performance Evaluations

Turfgrass performance evaluation ratings in multiple tests likewise distinguish Eclipse from other St. Augustinegrass varieties.

### University of Florida Test

Eclipse was first evaluated under the experimental name MSA 31 at the University of Florida in Gainesville, Fla. in the years 1995-1998 along with eleven (11) other St. Augustinegrass cultivars. Grasses were replicated four (4) times in a randomized complete block design. As shown in Tables 2 through 5, the ‘MSA’ cultivars were Mississippi St. Augustinegrass experimental cultivars from MSU. The ‘FHSA’ cultivars were Florida Hawaii St. Augustinegrass experimental cultivars collected in Hawaii and tested at the University of Florida. The ‘FL’ cultivar was a University of Florida experimental cultivar. The establishment of Eclipse was not as good as Raleigh, but generally better than Palmetto and Floratam, and significantly better than Seville in this test (Table 2). Eclipse received consistently high turf quality ratings (Table 3) and, in 1996, the turf quality rating of Eclipse was significantly higher than any other cultivar in the test. Turf density was rated for two years. Eclipse received higher turf density ratings than Floratam, Raleigh, Palmetto, and Seville in each year and, in fact, rated higher than all twelve cultivars tested in both years (Table 4). In 1998, this test rated leaf texture and seedhead production (Table 5). The leaf texture of Eclipse was finer than all but two other cultivars and significantly finer than Raleigh and Floratam. Seedhead production of Eclipse was less than all but one other cultivar and significantly less than the two MSA cultivars and Raleigh and Palmetto.

TABLE 2
Average percent ground cover estimates of St. Augustinegrass selections
during establishment at Gainesville, FL.

| | 1995 | | | 1996 | | | | | |
|---|---|---|---|---|---|---|---|---|---|
| Cultivar | Sept. | Oct. | Nov. | April | May | June | July | Aug. | Sept. |
| Raleigh | 26 | 31 | 33 | 41 | 52 | 81 | 92 | 93 | 97 |
| FL- | 28 | 29 | 31 | 42 | 51 | 74 | 90 | 93 | 95 |
| 1997-6 | | | | | | | | | |
| MSA- | 24 | 27 | 29 | 41 | 46 | 73 | 85 | 88 | 96 |
| 11 | | | | | | | | | |
| Eclipse | 22 | 23 | 25 | 32 | 42 | 73 | 84 | 89 | 95 |
| Pal- | 19 | 20 | 21 | 34 | 41 | 69 | 83 | 87 | 93 |
| metto | | | | | | | | | |
| MSA- | 15 | 18 | 23 | 35 | 42 | 69 | 82 | 87 | 91 |
| 10 | | | | | | | | | |
| Flora- | 12 | 17 | 21 | 34 | 42 | 64 | 80 | 86 | 92 |
| tam | | | | | | | | | |
| Flora- | 12 | 16 | 21 | 31 | 40 | 68 | 81 | 86 | 92 |
| lawn | | | | | | | | | |
| Flora- | 9 | 14 | 18 | 30 | 36 | 60 | 72 | 82 | 91 |
| tine | | | | | | | | | |
| FHSA- | 9 | 12 | 15 | 27 | 34 | 57 | 73 | 81 | 88 |
| 115 | | | | | | | | | |
| FSHA- | 8 | 10 | 13 | 24 | 31 | 55 | 71 | 81 | 91 |
| 117 | | | | | | | | | |
| Seville | 5 | 5 | 5 | 12 | 16 | 34 | 50 | 61 | 66 |
| MSD | 3 | 3 | 4 | 5 | 6 | 10 | 8 | 5 | 5 |
| (0.05) | | | | | | | | | |

TABLE 3
Turf quality ratings of St. Augustinegrass selections at Gainesville, FL.
(1 = poor, 9 = excellent)

| Cultivar | 1996 | 1997 | 1998 |
|---|---|---|---|
| Eclipse | 7.6 | 5.6 | 5.4 |
| FSHA-117 | 6.3 | 5.5 | 5.6 |
| MSA-l1 | 6.3 | 5.4 | 5.6 |
| Floratine | 6.2 | 6.0 | 5.9 |
| Floratam | 6.2 | 5.7 | 5.3 |
| FHSA-115 | 6.1 | 5.5 | 5.4 |
| FL-1997-6 | 5.9 | 6.0 | 6.9 |
| Floralawn | 5.9 | 5.5 | 5.1 |
| Raleigh | 5.4 | 4.6 | 4.6 |
| Palmetto | 5.2 | 5.2 | 5.5 |
| MSA-10 | 5.1 | 4.7 | 4.8 |
| Seville | 3.8 | 4.3 | 5.3 |
| MSD (0.05) | 1.0 | 1.0 | 0.9 |

TABLE 4
Turf density ratings of St. Augustinegrass selections at Gainesville, FL.
(1 = low, 9 = high)

| Cultivar | 1996 | 1997 |
|---|---|---|
| Eclipse | 8.6 | 7.3 |
| FL- 1997-6 | 7.3 | 7.2 |
| MSA-11 | 7.2 | 7.1 |
| FSHA-1 17 | 7.1 | 6.5 |
| Floratine | 6.9 | 6.5 |
| FHSA-1 15 | 6.8 | 6.2 |
| Floratam | 6.5 | 6.1 |
| Palmetto | 6.4 | 6.6 |
| Raleigh | 6.4 | 6.1 |
| Floralawn | 6.4 | 5.8 |
| MSA-10 | 6.2 | 6.2 |
| Seville | 5.3 | 5.7 |
| MSD (0.05) | 0.8 | 0.7 |

TABLE 5
Leaf texture and seedhead production of St. Augustinegrass selections
at Gainesville, FL in 1998. (Texture: 1 = coarse, 9 = fine)

| Cultivar | Leaf texture | Seedheads⁺ |
|---|---|---|
| MSA-10 | 6.8 a* | 5.0 a |
| Eclipse | 6.0 ab | 1.1 fg |
| FL-1997-6 | 6.0 ab | 1.9 de |
| MSA-11 | 5.8 bc | 4.1 bc |
| Seville | 5.5 bc | 1.0 g |
| Palmetto | 5.2 bcd | 3.5 c |
| Raleigh | 5.0 cd | 4.4 ab |
| FHSA-115 | 5.0 cd | 1.5 efg |
| Floratine | 4.5 de | 2.4 d |
| FSHA-117 | 3.8 ef | 1.8 def |
| Floratam | 3.0 f | 1.6 efg |
| Floralawn | 3.0 f | 1.5 efg |

⁺Seedheads visually rated on a 1 to 5 scale where 1 = no seedheads and 5 = most seedheads. Values are average of 8 observations.
*Means within columns with same letter are not significantly different (P = 0.05) using the Waller-Duncan k-ratio t-test.

### Lawrenceburg, Tenn. Test

A St. Augustinegrass cultivar trial was conducted at the Lawrence County Extension Center at Lawrenceburg, Tenn. in the years 2002-2005. Eight (8) St. Augustinegrass cultivars, including Raleigh, Floratam, Eclipse, and other experimental cultivars, were planted in a randomized complete block design with three (3) replications. Eclipse received high turf quality ratings in the Lawrenceburg test (Table 6). The cold tolerance of Eclipse shows lower percent green plot cover ratings in the spring, although its percent green plot cover rating in the fall of 2003 was exceptionally high (Table 7). Due to the severe winter of 2004-05, all test cultivars suffered winter injury as shown in the May 12, 2005 measurements of Table 7. Eclipse displayed good color and good turf density and color with moderate symptoms of gray leaf spot (Table 8). Eclipse demonstrated the best color rating of all cultivars tested and a better turf density rating than all but two of the cultivars in the test.

TABLE 6
Turf quality of St. Augustinegrass test at Lawrenceburg, TN.
(1 = poor, 9 = excellent)

| Cultivar | 2002 | 2003 | 2004 | 2005 | 2002-2005 |
|---|---|---|---|---|---|
| Aurora | 7.0 | 8.0 | 7.7 | 7.3 | 7.5 |
| MSA-10-4-98 | 6.7 | 7.3 | 7.6 | 6.0 | 6.9 |
| Eclipse | 7.0 | 8.0 | 6.4 | 5.3 | 6.7 |
| Raleigh | 6.0 | 7.3 | 6.5 | 5.7 | 6.4 |
| MSA-8-6-98 | 5.3 | 5.7 | 6.0 | 6.7 | 5.9 |
| MSA-5-5-98 | 5.3 | 4.7 | 5.3 | 5.3 | 5.2 |
| Floratam | 4.7 | 4.3 | 4.2 | 2.3 | 3.9 |
| MSA-13-6-98 | 4.3 | 2.3 | 3.5 | 4.3 | 3.6 |
| LSD (0.05) | 1.2 | 2.2 | 2.2 | 2.2 | 1.6 |

TABLE 7
Percent green plot cover of St. Augustinegrass test at Lawrenceburg, TN.

| | May 18, | Nov. 13, | May 5, | May 12, | Aug. 18, |
|---|---|---|---|---|---|
| Cultivar | 2003 | 2003 | 2004 | 2005 | 2005 |
| Aurora | 95.0 | 100.0 | 70.0 | 25.0 | 91.7 |
| Raleigh | 86.7 | 100.0 | 60.0 | 25.0 | 81.7 |
| MSA-10-4-98 | 71.7 | 95.0 | 50.0 | 6.7 | 81.7 |
| MSA-8-6-98 | 70.0 | 66.7 | 56.7 | 38.3 | 76.7 |
| MSA-13-6-98 | 50.3 | 16.7 | 43.3 | 21.7 | 51.7 |
| MSA-5-5-98 | 46.7 | 56.7 | 43.3 | 35.0 | 68.3 |
| Eclipse | 21.7 | 98.3 | 16.7 | 4.7 | 53.3 |
| Floratam | 3.7 | 53.3 | 3.3 | 2.7 | 20.0 |
| LSD (0.05) | 35.7 | 32.2 | 27.1 | 20.3 | 33.9 |

TABLE 8
Color, density, and gray leaf spot ratings of the St. Augustinegrass test
at Lawrenceburg, TN rated on Aug. 18, 2005.

| | | | |
|---|---|---|---|
| Cultivar | Color | Density | Gray Leaf Spot^{a} |

| | | | |
|---|---|---|---|
| Aurora | 7.3 | 7.0 | 8.0 |
| Eclipse | 7.7 | 6.0 | 5.0 |
| MSA-10-4-98 | 7.0 | 6.0 | 6.7 |
| Raleigh | 6.7 | 5.7 | 7.0 |
| MSA-8-6-98 | 6.0 | 5.7 | 6.7 |
| Floratam | 5.3 | 4.0 | 2.0 |
| MSA-5-5-98 | 6.0 | 3.7 | 5.7 |
| MSA-13-6-98 | 4.3 | 3.0 | 4.3 |
| LSD (0.05) | NS | 2.3 | 2.4 |

^{a}Gray leaf spot rating scale: 1 = severe, 9 = no disease
Color scale: 1 = light green, 9 = dark green
Density scale: 1 = low, 9 = high

### National Turfgrass Evaluation Program Test

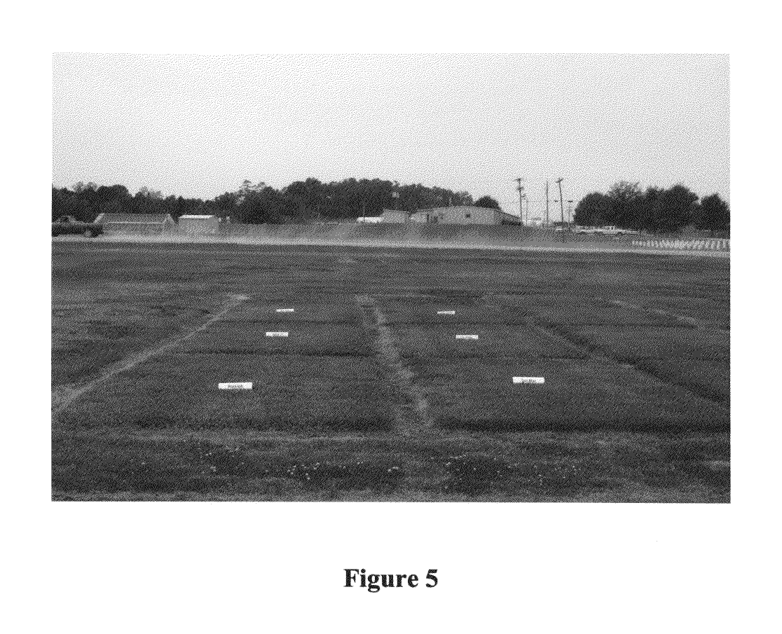

Eclipse was entered under the experimental name MSA 31 into the 2002 National St. Augustinegrass Test coordinated by the National Turfgrass Evaluation Program (NTEP). This multiple-environment testing provided the best available and the most definitive data on the performance characteristics of Eclipse compared to other commercial and experimental St. Augustinegrass cultivars. The nine (9) test sites included Pomona, Calif.; Jay, Fla.; Griffin, Ga.; Savannah, Ga.; Calhoun, La.; Mississippi State (Starkville), Miss.; Lane, Okla.; Florence, S.C.; and College Station, Tex. Each site test included each variety listed in Tables 9 through 29. The specified experimental design at each site was a randomized complete block with three (3) replications of each variety tested. All cultivars were established by planting eighteen (18) 3-inch plugs in each plot. At each site, Eclipse was compared to Aurora, Delmar, Floratam, Mercedes, and Raleigh. FIG. 5 shows field plots of Eclipse and five other cultivars in the 2002 NTEP test at the Starkville site.

Establishment rate was determined by estimating percent plot coverage at monthly intervals and dividing by the number of ratings to arrive at a mean. Table 9 represents the establishment rates of the six (6) cultivars at six (6) of the nine (9) test locations. Eclipse established slower from plugs than Floratam at each location due most likely to Eclipse's significantly shorter internode length.

Tests were established at three (3) locations (Calhoun, La.; Starkville, Miss.; and Lane, Okla.) and data was submitted on turfgrass quality ratings in full sun in 2002 (Table 10). Eclipse received the highest turfgrass quality rating of all cultivars except one at Calhoun and the best turfgrass quality rating at Lane, both in full sun. Its turfgrass quality ratings were significantly higher than Floratam at all three sites and significantly higher than Raleigh at Calhoun and Lane. Other sites were subsequently established and data was reported beginning in 2003 (Table 11). When considered across eight (8) sites where the cultivars were grown in full sun, Eclipse received a 4-year average turfgrass quality rating that was slightly higher than Raleigh, Floratam, and Delmar and not significantly lower than Aurora and Mercedes. As Table 11 shows, Eclipse ranked the highest in turfgrass quality at Pomona.

A dense shade site was planted at Savannah, Ga. in 2002 and turfgrass quality ratings data began to be reported early in 2003 (Table 12). Eclipse consistently ranked at or near the top for turfgrass quality under dense shade at this site, ranking the best for 2003, 2004, and 2006 and better than all but one cultivar for 2005. Eclipse had the highest mean turfgrass quality rating under dense shade for 2003-2006.

The exceptional turfgrass quality of Eclipse under dense shade is significant, particularly since the effects of shade generally cause lower turf density. As shown in Table 13, Eclipse maintained exceptionally high average turfgrass density ratings under dense shade at this site during both the summer and fall seasons.

Five (5) sites provided leaf texture ratings of St. Augustinegrass cultivars from 2003 to 2006 (Table 14). Eclipse was rated consistently fine textured at each site and was rated as having the finest leaf texture at Pomona from 2003 to 2005. When averaged across all five (5) sites over four (4) years, Eclipse ranked finer than all other cultivars tested.

Genetic color ratings should generally be conducted when turfgrass is actively growing and is not under stress in order to reflect the inherent color of the cultivar. Genetic color in this test was rated at seven (7) sites during 2003-2006. When averaged across all sites, the genetic color rating of Eclipse ranked in the middle and was not significantly different from the other cultivars, although it ranked as having one of the three best color ratings at the Pomona site (Table 15). Fall and winter color ratings are used to assess color retention during the fall and winter months, respectively. Table 16 and Table 17 show high fall and winter color retention of Eclipse at sites that did not experience much frost. The frost tolerance of Eclipse was lower at some test locations (Table 18).

Similar to the Lawrenceburg, Tenn. test evaluation data, Eclipse suffered winter injury at colder locations during the NTEP test. Percent winter kill estimates of Eclipse were high at the Lane and Florence sites (Table 19). Spring green-up of St. Augustinegrasses is affected by the amount of low-temperature injury in the winter. Eclipse was slower to green-up in the spring than some cultivars at colder test sites, including Griffin, Starkville, Lane, and Florence, but was the fastest or one of the fastest to green-up at warmer sites such as Pomona, Jay, and College Station (Table 20). Low temperatures also affected estimates of percent living ground cover in spring (Table 21). Eclipse displayed lower coverage in spring at the colder sites.

Eclipse was less susceptible than four (4) of the six (6) cultivars to warm temperature brown patch disease (Rhizoctonia solani) at the Florence and College Station sites (Table 22) and less susceptible than all other cultivars to cool temperature brown patch disease (Rhizoctonia cerealis) at the Florence site (Table 23). Gray leaf spot was not as severe on Eclipse as on other cultivars at test sites in Florida, Mississippi, and Oklahoma (Table 24). Eclipse was more susceptible than Raleigh and Floratam to take-all patch (Gaumanomyces graminis) at the Lane site in 2004 (Table 25).

The production of seedheads (inflorescence) in turf is undesirable. Seedhead production of the cultivars in the NTEP test was measured at the Lane site (Table 26) and the Starkville site (Table 27). At both sites, Eclipse produced fewer seedheads than both Raleigh and Delmar.

The horizontal spread rating of the St. Augustinegrass cultivars was rated at the Lane site in 2004 (Table 28). The horizontal spread of Eclipse was rated significantly slower than Floratam and, as with establishment rate, was likely due to the shorter internode length of Eclipse. Once established, however, Eclipse may require less frequent edging around sidewalks and beds.

Chinch bug (Blissus insularis) populations can be devastating on St. Augustinegrass turf. Floratam St. Augustinegrass generally has a high resistance to this small insect pest. Chinch bugs were counted at the NTEP St. Augustinegrass test at the Griffin site in 2006 (Table 29). Numbers of chinch bugs for Eclipse were significantly higher than Floratam but significantly lower than Raleigh.

TABLE 9
Establishment rate of cultivars in the 2002
National St. Augustinegrass Test.

| | | College | Stark- | | | |
|---|---|---|---|---|---|---|
| | Calhoun, | Station, | ville, | Pomona, | Jay, | Lane, |
| Cultivar | LA | TX | MS | CA | FL | OK |
| Floratam | 83.3% | 78.3% | 81.6% | 92.6% | 41.7% | 92.2% |
| Raleigh | 63.3 | 60.0 | 81.9 | 88.1 | 17.2 | 77.6 |
| Aurora | 71.7 | 36.7 | 73.1 | 74.7 | 41.7 | 85.2 |
| Mercedes | 73.3 | 48.3 | 76.7 | 81.0 | 15.0 | 73.9 |
| Eclipse | 70.0 | 28.3 | 73.3 | 78.1 | 13.0 | 84.7 |
| Delinar | 46.7 | 36.7 | 69.2 | 79.6 | 12.8 | 63.3 |
| LSD | 16.6 | 21.4 | 7.9 | 17.0 | 5.8 | 6.5 |

TABLE 10
Turfgrass quality ratings from the 2002 National St. Augustinegrass Test
established at 3 U.S. locations in full sun.
(2002 Data: 1 = poor, 9 = excellent)

| | Calhoun, | Starkville, | Lane, |
|---|---|---|---|
| Cultivar | LA | MS | OK |
| Eclipse | 8.0 | 6.4 | 8.0 |
| Aurora | 8.0 | 6.9 | 7.5 |
| Mercedes | 7.7 | 6.6 | 5.7 |
| Raleigh | 7.0 | 6.3 | 5.7 |
| Floratam | 6.0 | 5.4 | 7.3 |
| Delmar | 6.3 | 6.2 | 4.5 |
| LSD | 0.8 | 0.3 | 0.5 |

TABLE 11
Turfgrass quality ratings from the 2002 National St. Augustinegrass Test grown
at 8 U.S. locations in full sun.
(1 = poor, 9 = excellent)

| | Ponoma, | Jay, | Griffin, | Calhoun, | Starkville, | Lane, | Florence, | College Station, | |
|---|---|---|---|---|---|---|---|---|---|
| | CA | FL | GA | LA | MS | OK | SC | TX | |
| Cultivar | 2003-2006 | 2003-2006 | 2004-2006 | 2003-2006 | 2003-2006 | 2003-2006 | 2003-2006 | 2003-2004 | Mean |
| Aurora | 6.2 | 5.4 | 6.8 | 6.8 | 7.5 | 6.1 | 6.1 | 5.0 | 6.3 |
| Mercedes | 6.4 | 5.9 | 7.5 | 6.8 | 7.1 | 5.3 | 6.0 | 5.3 | 6.3 |
| Eclipse | 6.6 | 5.4 | 6.8 | 6.8 | 6.4 | 5.1 | 5.5 | 4.8 | 6.0 |
| Raleigh | 5.8 | 5.2 | 6.6 | 6.6 | 6.5 | 6.0 | 5.4 | 4.9 | 5.9 |
| Floratam | 6.1 | 6.1 | 6.3 | 6.3 | 4.5 | 6.6 | 5.1 | 5.8 | 5.8 |
| Delmar | 5.8 | 4.8 | 7.0 | 6.4 | 6.3 | 5.8 | 5.2 | 4.6 | 5.8 |
| LSD | 1.0 | 1.1 | 1.1 | 0.6 | 0.4 | 1.3 | 0.9 | 0.8 | 0.4 |

TABLE 12
Turfgrass quality ratings from the 2002 National
St. Augustinegrass Test grown under dense shade at Savannah, GA.
(1 = poor, 9 = excellent)

| Cultivar | 2003 | 2004 | 2005 | 2006 | Mean |
|---|---|---|---|---|---|
| Eclipse | 7.2 | 7.6 | 7.6 | 6.6 | 7.2 |
| Raleigh | 6.6 | 7.5 | 6.8 | 6.2 | 6.7 |
| Aurora | 6.3 | 7.4 | 7.8 | 5.7 | 6.6 |
| Mercedes | 6.0 | 6.9 | 6.8 | 5.8 | 6.4 |
| Delmar | 5.5 | 7.1 | 6.8 | 6.0 | 6.3 |
| Floratam | 4.7 | 5.5 | 5.3 | 4.5 | 5.0 |
| LSD | 0.9 | 0.7 | 1.1 | 1.6 | 0.6 |

TABLE 13
Density ratings from the 2002 National St. Augustinegrass
Test grown under dense shade at Savannah, GA.
(1 = low, 9 = high)

| | Spring density | Summer density | Fall density |
|---|---|---|---|
| Cultivar | 2004-06 | 2004-06 | 2004-05 |
| Eclipse | 6.2 | 7.0 | 7.2 |
| Aurora | 6.7 | 6.8 | 7.3 |
| Raleigh | 6.8 | 6.2 | 5.8 |
| Mercedes | 5.9 | 5.8 | 5.8 |
| Delmar | 5.6 | 6.2 | 6.3 |
| Floratam | 3.3 | 4.2 | 4.5 |
| LSD | 1.7 | 1.1 | 1.5 |

TABLE 14
Leaf texture ratings from the 2002 National St. Augustinegrass Test.
(1 = coarse, 9 = fine)

| | | | Lane, | | College | |
|---|---|---|---|---|---|---|
| | Ponoma, | Starkville, | OK | Florence, | Station, | |
| | CA | MS | 2004- | SC | TX | |
| Cultivar | 2003-2005 | 2003-2006 | 2006 | 2003-2005 | 2003 | Mean |
| Eclipse | 5.4 | 7.8 | 5.7 | 2.0 | 6.0 | 5.6 |
| Aurora | 5.0 | 6.8 | 5.4 | 2.0 | 5.7 | 5.1 |
| Mercedes | 5.0 | 6.8 | 5.7 | 1.7 | 6.0 | 5.1 |
| Raleigh | 4.9 | 5.8 | 5.4 | 1.3 | 5.0 | 4.7 |
| Delmar | 4.8 | 5.9 | 4.4 | 1.0 | 5.7 | 4.4 |
| Floratam | 3.9 | 4.4 | 4.7 | 1.7 | 4.0 | 4.0 |
| LSD | 1.6 | 0.4 | 0.7 | 0.0 | 0.5 | 0.5 |

TABLE 15
Genetic color ratings from the 2002 National St. Augustinegrass Test.
(1 = light green, 9 = dark green)

| | Ponoma, | Jay, | Griffin, | Starkville, | Lane, | Florence, | College Station, | |
|---|---|---|---|---|---|---|---|---|
| | CA | FL | GA | MS | OK | SC | TX | |
| Cultivar | 2003-2006 | 2003-2006 | 2004-2006 | 2003-2006 | 2003-2005 | 3003-2005 | 2003 | Mean |
| Aurora | 5.8 | 5.9 | 7.4 | 7.6 | 7.2 | 6.3 | 4.7 | 6.5 |
| Mercedes | 6.5 | 6.2 | 7.9 | 7.1 | 5.9 | 6.0 | 4.7 | 6.5 |
| Delmar | 6.3 | 5.0 | 7.7 | 7.2 | 7.4 | 6.2 | 5.0 | 6.5 |
| Eclipse | 6.5 | 5.8 | 7.8 | 7.4 | 6.6 | 5.3 | 4.7 | 6.4 |
| Raleigh | 5.9 | 5.2 | 7.6 | 6.8 | 6.3 | 5.8 | 4.7 | 6.1 |
| Floratam | 6.5 | 6.1 | 6.7 | 7.0 | 5.1 | 5.1 | 6.0 | 6.1 |
| LSD | 1.6 | 1.0 | 1.1 | 0.7 | 0.8 | 0.9 | 0.8 | 0.4 |

TABLE 16
Fall Color (November) ratings from the
2002 National St. Augustinegrass Test.
(1 = brown, 9 = completely green)

| | | | | Lane, | | |
|---|---|---|---|---|---|---|
| | Ponoma, | Jay, | Griffin, | OK | Florence, | |
| | CA | FL | GA | 2003- | SC | |
| Cultivar | 2003-2005 | 2003-2005 | 2004 | 2004 | 2004 | Mean |
| Eclipse | 5.7 | 4.3 | 5.3 | 7.2 | 6.0 | 5.5 |
| Floratam | 4.9 | 5.0 | 4.3 | 7.7 | 5.0 | 5.4 |
| Delmar | 5.2 | 4.0 | 6.3 | 6.7 | 5.0 | 5.2 |
| Mercedes | 5.1 | 4.4 | 6.0 | 5.3 | 5.3 | 5.0 |
| Aurora | 4.7 | 4.2 | 5.3 | 5.8 | 4.7 | 4.8 |
| Raleigh | 4.2 | 4.2 | 5.3 | 4.7 | 4.7 | 4.5 |
| LSD | 1.2 | 1.0 | 1.1 | 1.7 | 2.3 | 0.7 |

TABLE 17
Winter color ratings from the 2002 National St. Augustinegrass Test.
(1 = brown, 9 = completely green)

| | Pomona, | Jay, | College Station, | |
|---|---|---|---|---|
| | CA | FL | TX | |
| Cultivar | 2005 | 2005 | 2003 | Mean |
| Eclipse | 5.3 | 2.3 | 4.7 | 4.3 |
| Floratam | 5.3 | 3.0 | 3.3 | 3.8 |
| Mercedes | 5.3 | 3.0 | 3.0 | 3.6 |
| Aurora | 4.7 | 2.0 | 3.3 | 3.3 |
| Raleigh | 4.7 | 3.0 | 2.7 | 3.3 |
| Delmar | 4.7 | 2.3 | 2.3 | 2.9 |
| LSD | 1.7 | 1.3 | 1.4 | 1.2 |

TABLE 18
Frost tolerance ratings from the 2002 National St. Augustinegrass Test.
(1 = brown, 9 = completely green)

| | Griffin, | Starkyille, | Lane, | |
|---|---|---|---|---|
| | GA | MS | OK | |
| Cultivar | 2004 | 2005-2006 | 2003-2006 | Mean |
| Aurora | 3.3 | 7.2 | 6.3 | 6.2 |
| Mercedes | 2.3 | 7.0 | 6.4 | 6.0 |
| Raleigh | 4.0 | 6.5 | 5.7 | 5.6 |
| Eclipse | 2.3 | 5.0 | 5.9 | 5.3 |
| Delmar | 2.3 | 5.0 | 5.9 | 5.1 |
| Floratam | 6.7 | 3.2 | 4.3 | 4.3 |
| LSD | 2.4 | 1.1 | 1.3 | 1.0 |

TABLE 19
Percent winterkill estimates from the
2002 National St. Augustinegrass Test.

| | Lane, | Florence, | |
|---|---|---|---|
| | OK | SC | |
| Cultivar | 2003-2006 | 2003 | Mean |

| | | | |
|---|---|---|---|
| Eclipse | 57.1 | 43.3 | 60.4 |
| Floratam | 55.0 | 70.0 | 58.3 |
| Mercedes | 40.8 | 6.7 | 39.2 |
| Aurora | 39.2 | 2.3 | 38.6 |
| Delmar | 36.3 | 16.7 | 36.4 |
| Raleigh | 33.8 | 11.7 | 34.2 |
| LSD | 17.4 | 14.9 | 17.7 |

TABLE 20
Spring green-up ratings from the 2002 National St. Augustinegrass Test during 2003-2006.
(1 = brown, 9 = completely green)

| | Ponoma, | Jay, | Griffin, | Calhoun, | Starkville, | Lane, | Florence, | College Station, | |
|---|---|---|---|---|---|---|---|---|---|
| | CA | FL | GA | LA | MS | OK | SC | TC | |
| Cultivar | 2004-2005 | 2003-2005 | 2005 | 2003-2004 | 2003-2006 | 2003-2006 | 2003-2005 | 2004 | Mean |
| Mercedes | 7.2 | 4.6 | 4.3 | 6.2 | 5.6 | 4.1 | 3.7 | 2.7 | 4.8 |
| Aurora | 7.0 | 4.1 | 5.0 | 6.0 | 6.2 | 3.1 | 3.8 | 2.7 | 4.7 |
| Delmar | 5.7 | 4.8 | 4.7 | 6.2 | 4.3 | 4.5 | 3.7 | 3.3 | 4.5 |
| Raleigh | 6.3 | 4.8 | 4.3 | 6.0 | 5.0 | 3.3 | 3.0 | 2.0 | 4.5 |
| Eclipse | 7.8 | 5.4 | 2.0 | 5.2 | 3.1 | 2.2 | 2.9 | 4.0 | 3.7 |
| Floratam | 6.2 | 5.1 | 2.0 | 6.0 | 1.9 | 2.6 | 2.2 | 4.0 | 3.5 |
| LSD | 1.7 | 1.1 | 1.5 | 1.2 | 1.0 | 1.1 | 0.9 | 0.7 | 0.6 |

TABLE 21
Percent living ground cover in spring from the 2002 National St. Augustinegrass Test.

| | Pomona, | Calhoun, | Starkville, | Lane, | Florence, | College Station, | |
|---|---|---|---|---|---|---|---|
| | CA | LA | MS | OK | SC | TX | |
| Cultivar | 2004-2005 | 2003 | 2003-2006 | 2003-2006 | 2003 | 2003-2004 | Mean |
| Raleigh | 96.8% | 76.7% | 85.8% | 63.2% | 5.0% | 84.7% | 74.1% |
| Mercedes | 94.3 | 80.0 | 89.4 | 58.3 | 11.0 | 83.0 | 73.4 |
| Aurora | 90.2 | 78.3 | 93.1 | 60.4 | 16.0 | 78.7 | 72.9 |
| Delmar | 87.3 | 70.0 | 76.7 | 58.8 | 15.7 | 67.3 | 66.5 |
| Eclipse | 92.8 | 75.0 | 57.9 | 41.0 | 4.7 | 71.3 | 56.4 |
| Floratam | 94.5 | 75.0 | 37.9 | 42.3 | 1.7 | 92.0 | 55.5 |
| LSD | 10.3 | 7.1 | 11.8 | 17.4 | 7.9 | 19.6 | 9.7 |

TABLE 22
Warm temperature brown patch disease (Rhizoctonia solani)
rating^{a} from the 2002 National St. Augustinegrass Test.

| | Florence, SC | College Station, TX | |
|---|---|---|---|
| Cultivar | 2004 | 2003-2004 | Mean |
| Floratam | 8.3 | 7.8 | 8.3 |
| Eclipse | 8.3 | 7.8 | 8.2 |
| Aurora | 6.7 | 7.2 | 7.3 |
| Mercedes | 6.3 | 7.0 | 6.7 |
| Raleigh | 4.3 | 5.8 | 5.6 |
| Delmar | 5.0 | 5.3 | 4.8 |
| LSD | 3.3 | 4.2 | 3.0 |

^{a}Disease rating, 1 = severe, 9 = nodisease

TABLE 23
Cool temperature brown patch disease (Rhizoctonia cerealis) rating^{a} from
the 2002 National St. Augustinegrass Test.

| | Florence, SC |
|---|---|
| Cultivar | 2004-2005 |
| Eclipse | 7.5 |
| Floratam | 6.8 |
| Delmar | 6.3 |
| Aurora | 5.8 |
| Mercedes | 5.2 |
| Raleigh | 3.7 |
| LSD | 2.4 |

^{a}Disease rating, 1 = severe, 9 = no disease

TABLE 24
Gray leaf spot ratings from the 2002 National St. Augustinegrass Test.

| | Jay, FL | Starkville, MS | Lane, OK | |
|---|---|---|---|---|
| | 2003 | 2005 | 2003 | Mean |
| Eclipse | 8.0 | 6.7 | 8.0 | 7.3 |
| Aurora | 8.0 | 4.3 | 5.7 | 7.1 |
| Delmar | 6.7 | 6.7 | 6.0 | 6.5 |
| Raleigh | 4.0 | 7.7 | 6.3 | 6.4 |
| Mercedes | 4.0 | 7.0 | 6.3 | 6.1 |
| Floratam | 6.7 | 4.0 | 8.3 | 5.8 |
| LSD | 2.1 | 1.0 | 1.0 | 1.1 |

Gray leaf spot rating scale: 1 = severe, 9 = no disease

TABLE 25
Take-all patch rating at Lane, OK in 2004.

| | Lane, OK |
|---|---|
| Cultivar | 2004 |
| Floratam | 7.3 |
| Aurora | 7.0 |
| Delmar | 6.0 |
| Raleigh | 5.7 |
| Eclipse | 4.3 |
| Mercedes | 4.0 |
| LSD | 0.9 |

^{a}Disease rating, 1 = severe, 9 = no disease

TABLE 26
Seedhead rating of St. Augustinegrass cultivars at Lane, OK.

| Cultivar | 2003 | 2004 | 2005 | 2006 | Mean |
|---|---|---|---|---|---|
| Eclipse | 6.0 | 7.3 | 9.0 | 6.3 | 7.2 |
| Mercedes | 6.7 | 7.0 | 6.0 | 6.0 | 6.4 |
| Aurora | 4.3 | 5.3 | 6.7 | 8.7 | 6.3 |
| Floratam | 8.7 | 5.7 | 4.7 | 4.3 | 5.8 |
| Raleigh | 4.7 | 4.0 | 4.0 | 5.7 | 4.6 |
| Delmar | 4.3 | 2.7 | 3.3 | 6.7 | 4.3 |
| LSD | 1.1 | 1.2 | 1.5 | 2.9 | 1.7 |

Seedhead rating: 9 = no seedheads

TABLE 27
Seedhead rating of St. Augustinegrass cultivars at Starkville, MS.

| | Starkville, MS |
|---|---|
| Cultivar | 2003 |
| Mercedes | 8.0 |
| Eclipse | 7.7 |
| Floratam | 7.7 |
| Aurora | 7.3 |
| Delmar | 6.0 |
| Raleigh | 3.3 |
| LSD | 1.0 |

Seedhead rating: 9 = no seedheads

TABLE 28
Horizontal spread rating of St. Augustinegrass cultivars.

| | Lane, OK |
|---|---|
| Cultivar | 2004 |
| Floratam | 6.3 |
| Mercedes | 4.7 |
| Aurora | 4.3 |
| Raleigh | 4.3 |
| Delmar | 3.3 |
| Eclipse | 3.3 |
| LSD | 2.1 |

Spread rating: 1 = slow, 9 = fast

TABLE 29
Chinch bug counts on St. Augustinegrass cultivars at Griffin, GA in 2006.

| | August | August | December | December | |
|---|---|---|---|---|---|
| Cultivar | adult | nymph | adult | nymph | Mean |
| Raleigh | 70.7 | 13.3 | 65.7 | 151.7 | 75.3 |
| Aurora | 18.0 | 10.0 | 15.7 | 52.3 | 24.0 |
| Eclipse | 25.7 | 6.7 | 6.0 | 24.3 | 15.7 |
| Mercedes | 20.0 | 8.3 | 0.0 | 8.3 | 9.2 |
| Delmar | 12.7 | 0.7 | 0.0 | 5.3 | 4.7 |
| Floratam | 7.0 | 0.3 | 0.0 | 0.3 | 1.9 |
| LSD | 23.5 | 11.8 | 47.3 | 47.9 | 19.8 |

### SUMMARY

Observations and analyses on a comparative basis have identified specific characteristics of Eclipse that distinguish it from other St. Augustinegrasses including Raleigh, Floratam, Mercedes, Delmar, and various experimental cultivars. Eclipse has excellent turfgrass quality ratings and, particularly, superior quality and density ratings in shade and dense shade that allow it to be produced, marketed, and commercialized as a high quality St. Augustinegrass capable of excellent quality and density ratings in shade in warn climates and in United States Department of Agriculture (USDA) plant hardiness zones 8 and higher. Its superior leaf texture, genetic color, and fall and winter color ratings provide additional distinctions and advantages compared to other St. Augustinegrass cultivars.

As will be apparent to those skilled in horticultural science, the new and distinct perennial St. Augustinegrass variety described herein may vary in minor detail due to climatic, soil, and cultural conditions under which the variety may be grown, as well as the stage of growth.

### Comparative DNA Analysis of Eclipse with Other Turfgrasses

Randomly Amplified Polymorphic DNA (RAPD) analysis was used to generate DNA fingerprints that uniquely identified Eclipse from other St. Augustinegrasses. All tests and analyses were performed by Juliet D. Tang, Life Sciences and Biotechnology Institute (Mississippi State University).

### Plants

St. Augustinegrasses were obtained from Wayne Philley at Mississippi State University. Six turfgrass varieties were tested: Sapphire™, Raleigh, Aurora, Eclipse, Palmetto™, and Floratam. Grasses were grown individually in pots in the Institute greenhouse. One block consisted of each plant variety and each block was replicated four (4) times.

### DNA Isolation

DNA was extracted from young leaf blades according to the DNeasy Plant Mini Kit Protocol sold by Qiagen Inc. (Valencia, Calif.). Plant tissue (100 mg) was pulverized in liquid N2 using a mortar and pestle. Negative controls were subjected to the same procedures, except no plant tissue was added. After extraction, the DNA concentration was determined and all samples were diluted to the same working concentration.

### Amplification Primers

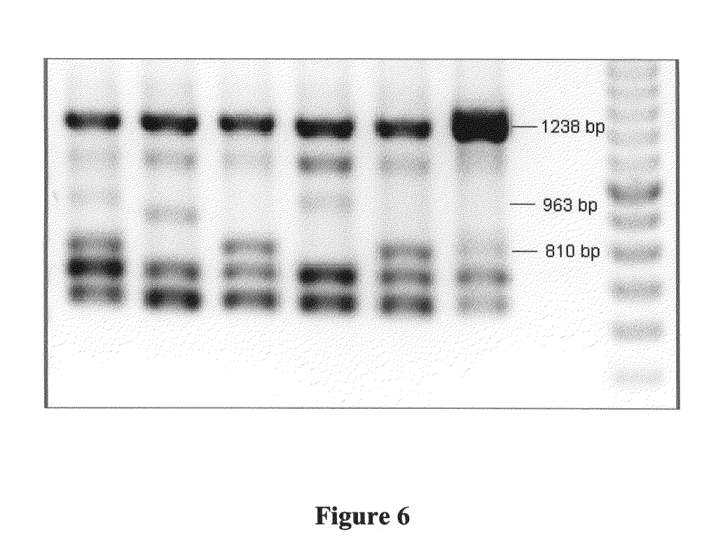

Eighty-five ten-mer primers were purchased from Operon Biotechnologies, Inc. (Huntsville, Ala.). Forty-four primers (OPA9, OPAA15, OPAA16, OPAB2, OPAC2, OPAC3, OPAC10, OPAC11, OPAC20, OPAE6, OPAF7, OPAG17, OPAH20, OPAI18, OPAN6, OPAO5, OPAP15, OPAT13, OPAU1, OPAV1, OPAX9, OPB4, OPB9, OPB10, OPB12, OPB15, OPB17, OPBA9, OPBE8, OPBE17, OPBG2, OPC4, OPC8, OPC11, OPC12, OPE2, OPH4, OPJ10, OPK4, OPM5, OPP15, OPP19, OPW15, OPY20) produced DNA fragments that were polymorphic, i.e. band(s) present in one variety and absent in another when tested against one block of grass DNA. Fifteen of these primers (OPA9, OPAC2, OPAC20, OPAH20, OPAI18, OPAU1, OPAX9, OPB4, OPB9, OPB12, OPBA9, OPC4, OPE2, OPH4, OPM5) were then tested against all four blocks of grass DNA. Primers OPAC20, OPAU1, OPBA9, and OPM5 produced consistent and unique fingerprints for Eclipse across all four replicates. An example fingerprint for each primer is exhibited in FIGS. 6 through 9, respectively.

### Amplification

Primer-specific amplification of DNA was performed using Takara Taq DNA polymerase (Takara Mirus Bio, Madison, Wis.), the manufacturer's supplied buffer, and 2.5 mM MgCl₂ in a polymerase chain reaction (PCR). The hot start method of preheating the DNA (25 ng) five minutes at 95 deg Celsius (C.) prior to the addition of the enzyme-primer master mix was employed. All amplifications were placed in a MyCycler thermal cycler (Bio-Rad, Hercules, Calif.) and run with the following program:

1. 2min at 95 deg C.

2. 30 s at 94 deg C.

3. 1 min at 35 deg C.

4. 2 min at 72 deg C.

5. return to step 2 and cycle 45 times

6. 20 min hold at 60 deg C.

7. infinite hold at 4 deg C.

### Gel Electrophoresis and Photography

DNA fragments produced by PCR were separated on a 1.5% agarose gel in Tris-acetate-EDTA buffer, then stained with EtBr₂, and visualized using a Versadoc 3000 (Bio-Rad, Hercules, Calif.). The lanes of the gel photographs, shown in FIGS. 6 through 9, correspond to (from left to right): Lane 1=Sapphire™, Lane 2=Raleigh, Lane 3=Aurora, Lane 4=Eclipse, Lane 5=Palmetto™, Lane 6=Floratam, Lane 7=negative control, and Lane 8=100 bp PCR molecular ruler size marker (Bio-Rad, Hercules, Calif.).

### RAPD Analysis

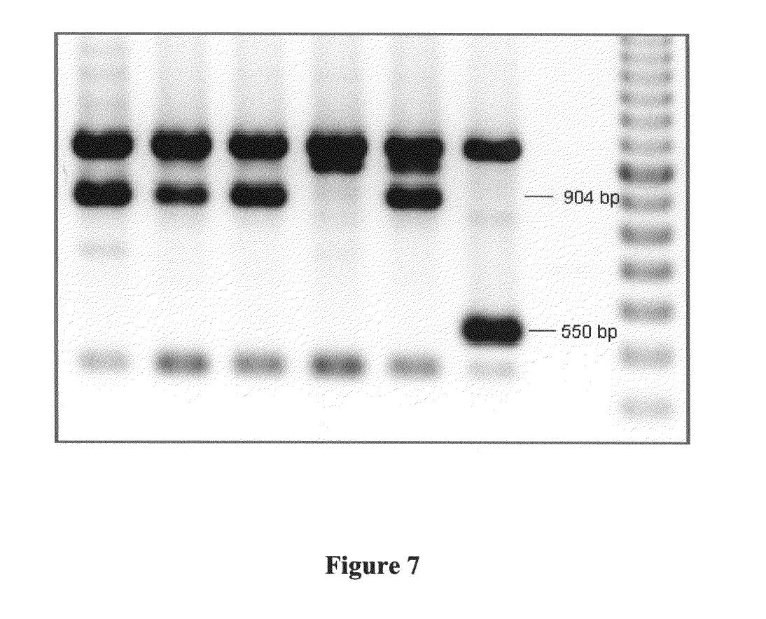

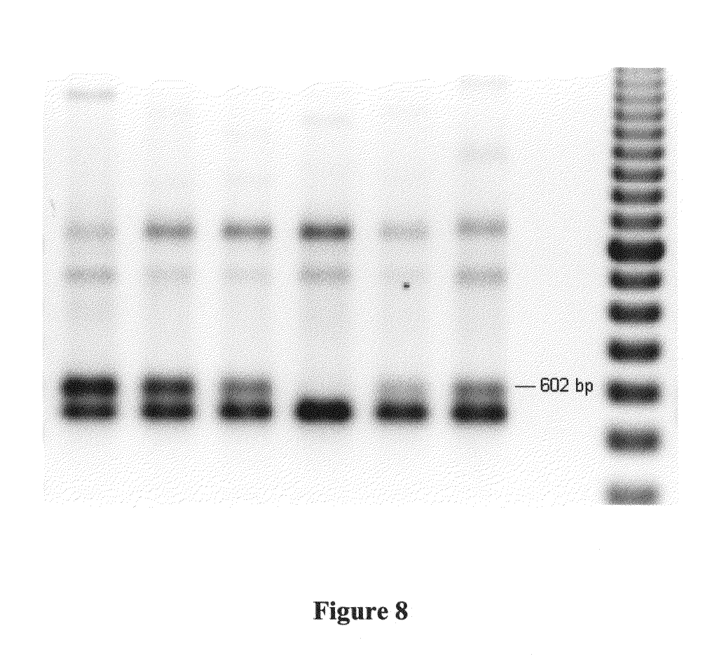

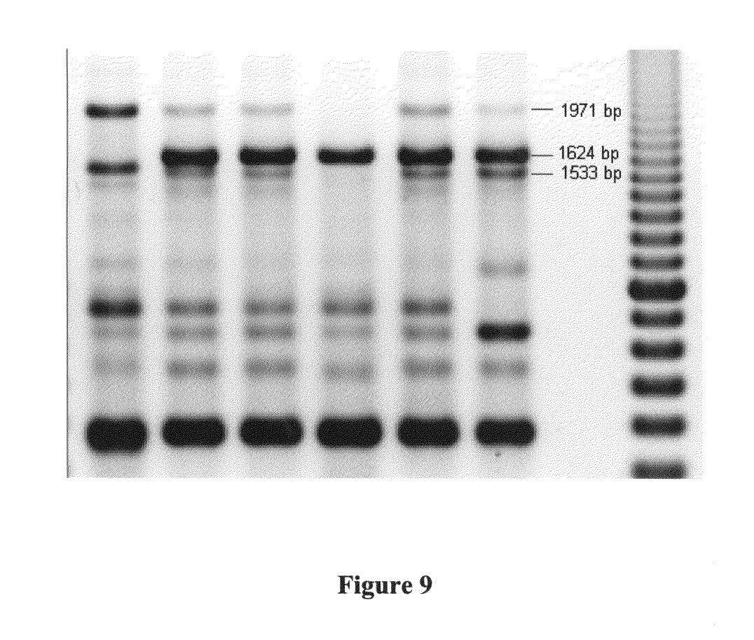

For RAPD analysis, bands in the gel images were detected, matched, and sized using QuantityOne software, (Bio-Rad, Hercules, Calif.). All samples could be distinguished from the others using one or more of the ten-mer primers. As shown in FIG. 6, Eclipse (Lane 4) shows the presence of the 963 bp band and the absence of the 810 bp band when tested with primer OPAC20. These two bands differentiate Eclipse from the other five cultivars tested. As shown in FIG. 7, Eclipse (Lane 4) shows absences of the 904 bp and the 550 bp bands when tested with primer OPAU1. These two bands differentiate Eclipse from the other five cultivars tested. As shown in FIG. 8, Eclipse shows the absence of the 602 bp band when tested with primer OPBA9. This band differentiates Eclipse from the other five cultivars tested. As shown in FIG. 9, Eclipse shows absences of the 1971 bp and the 1533 bp bands when tested with primer OPM5. These bands differentiate Eclipse from the other five cultivars tested.

What is claimed is:

1. A new and distinct variety of perennial St. Augustinegrass plant, substantially as herein illustrated and described.
